(19) United States
(12) Patent Application Publication
(10) Pub. No.: US 2021/0254123 A1
(43) Pub. Date: Aug. 19, 2021

(54) METHOD FOR THE DETECTION OF MICROORGANISMS AND DISK-SHAPED SAMPLE CARRIERS

(71) Applicant: Testo bioAnalytics GmbH, Titisee-Neustadt (DE)

(72) Inventor: Joel RIEMER, Breitnau (DE)

(73) Assignee: Testo bioAnalytics GmbH, Titisee-Neustadt (DE)

(21) Appl. No.: 17/173,488

(22) Filed: Feb. 11, 2021

(30) Foreign Application Priority Data
Feb. 14, 2020 (DE) 102020103963.8

(51) Int. Cl. C12Q 1/04

(52) U.S. Cl. CPC C12Q 1/04

(57) ABSTRACT

A detection method (1) for antibiotic-resistant microorganisms (2), in which at least one specific substance (3) which is broken down by an antibiotic resistance-causing enzyme (8) of the microorganism (2) is added to the sample (5), the enzyme (8) present triggers a reaction (4), and the reaction (4) involves generation of an optically detectable reaction product (6) near the resistant microorganism (2) that is subsequently detected in an optical detection method (7).

### INCORPORATION BY REFERENCE

German Patent Application No. DE 10 2020 103 963.8, filed Feb. 14, 2020, is incorporated herein by reference as if fully set forth.

### TECHNICAL FIELD

The invention relates to a method for detecting microorganisms and/or the properties thereof, wherein at least one specific substance which triggers a reaction of the microorganism to be detected is added to a sample in which the microorganisms are to be detected, wherein the reaction involves generation of an optically detectable reaction product which is subsequently detected in an optical detection method.

### BACKGROUND

Different methods of the kind mentioned at the start are already known, which are, for example, used for detection of antibiotic-resistant bacteria especially in hospitals, for example in human diagnostics.

In connection with this, culturing-based methods for example are known. However, they have the disadvantage that they require a large amount of time until the test result is established, since they are dependent on many necessary cell-division steps of the microorganisms. Moreover, it is not possible to perform corrective measures, such as targeted countermeasures against the microorganisms such as, for example, administration of the correct antibiotic, while carrying out the method, i.e., until the result is available.

Furthermore, rapid measurement methods based on using DNA or RNA for detection of certain properties (such as, for example, antibiotic resistance) are already known. Said methods are suitable for detecting the genetic disposition of the microorganisms when, for example, a resistance gene is being detected. However, they do not provide any detection of the actual phenotypical manifestation of a property, since the detection is not done at the protein level, i.e., via the phenotypical manifestation or the actual presence of, for example, resistance proteins. Furthermore, nucleic acid-based detection methods have the disadvantage that, in the event of mutations occurring in the target sequences of the DNA/RNA, the target sequences are no longer recognized; however, resistance may nevertheless be present and a false-negative result is thus generated. Other phenotypical detection methods have in turn the disadvantage that they require a high number of microorganisms for determination in order to generate a sufficiently high signal that can be detected, such as, for example, color changes around microorganism colonies on agar plates. Here, a large amount of time is therefore again required until completion of an analysis.

Other rapid measurement methods are merely indicative and not quantitative. Moreover, they do not provide a way to distinguish between living microorganisms and dead microorganisms.

### SUMMARY

It is therefore an object to provide an improved method for detecting microorganisms of the kind mentioned at the start, in which the disadvantages of previously known methods are overcome.

According to the invention, this object is achieved by a method having one or more of the features disclosed herein.

In particular, what is proposed according to the invention for achievement of the object is a method of the kind mentioned at the start, in which the reaction product is kept in spatial proximity to the reacting microorganism until optical detection. The method offers the advantages of a very low detection limit being achievable by detection of the presence of individual microorganisms by enriching the reaction product near the microorganism, thus yielding a stronger signal which is more easily detectable than with hitherto available methods. The method is therefore also safer than methods which are dependent on culturing and propagation of the microorganisms. The procedure can therefore be done in-house, i.e., without a laboratory, on-site and without laboratory training, since microorganisms do not need to be enriched. There is therefore no risk of contamination for humans and the environment.

Moreover, an absolute quantification of the positive microorganisms is possible, since RNA and/or DNA detection is not performed; instead, detection is done at the protein level and said detection can, for example, be assigned to even individual organisms. Therefore, a genuine statement can be made about the presence of a particular phenotype of a microorganism in a sample. The method furthermore has a shorter analysis time compared to previously known methods, meaning that it may be possible to treat affected patients more rapidly. Due to the ease of performance and to the fast analysis time of the method, it is particularly suitable for being performed without a laboratory and on-site. It can thus be used in hospitals, especially even in hospital wards. The method is moreover more cost-effective than laboratory-based methods, since equipment and performance steps can be dispensed with. The system can be used for detection of antibiotic resistances, but can equally be used for the detection of other properties of microorganisms.

In the case of the detection of a bacterium, the specific substance can, for example, be chemically bound to a cell wall and/or an envelope of the bacterium.

Advantageous configurations of the invention will be described below, which can be combined alone or in combination with the features of other configurations optionally together with the features noted above.

In addition to optical evaluation, it is also possible to use biochemical methods for detection.

According to an advantageous development, the reaction product can bind to an outer surface of the microorganism. What can therefore be particularly effectively ensured is that the reaction product does not separate from the microorganism, which, for example, could lead to yielding of an incorrect analysis result. For example, this allows better local enrichment of a reaction product near or within a microorganism until a detection threshold is reached that leads to a positive detection result. Moreover, the spatial concentration means that the period of time that must be waited for in order to be able to carry out the detection is shorter. Moreover, the sensitivity of the method can be increased, since even individual positive microorganisms are capturable. The advantage of the spatial concentration is that a detectable concentration of a reaction product can be reached more rapidly than if the product is distributed in a relatively large volume (e.g., in a relatively large reaction chamber). The reaction can therefore be carried out more rapidly, with less background noise arising at the same time.

For example, the binding of the reaction product to the microorganism can be achieved via a mediator, such as, for example, an antibody to which the specific substance has been bound. As a result of binding of the specific antibody to an antigen of the microorganism, the specific substance and/or the reaction product can be held and/or concentrated locally on the outer side of the microorganism. Moreover, the reagent can also be designed such that the resultant reaction product is highly reactive and immediately reacts with its environment (e.g., with the envelope of the microorganisms) and is thus fixed spatially close to the reaction site.

According to a further advantageous development, the individual microorganisms can be surrounded by a phase boundary in relation to an outer carrier fluid. In particular, the carrier fluid which can be used is a preferably hydrophobic liquid, such as oil, or air. In particular, the reaction product can be enclosed by the phase boundaries. This configuration is an additional and/or alternative configuration in relation to the configuration described in the preceding paragraph. The advantages are the same, since what is made possible here as well is better local enrichment of the reaction product near the microorganism and prevention of dissociation of the reaction product. Said phase boundary can, for example, be achieved by introducing the microorganisms, admixed with the substance (substrate), into a carrier liquid and/or a carrier fluid through a nozzle, it being possible for the nozzle to be designed as an atomizer nozzle. It may be particularly expedient when the microorganisms, especially together with the specific substance, are introduced into droplets. The microorganisms can, for example, be kept in an aqueous environment.

According to an advantageous configuration, a sensitive range of the detection method can have been adjusted to a dimension of the microorganisms. Individual capturing of microorganisms is therefore simple to carry out, for example for counting.

To be able to form especially uniformly sized droplets in which one microorganism or multiple microorganisms is/are enclosed, a phase boundary surrounding the individual microorganisms, for example the phase boundary already mentioned above, can be generated by spraying of the microorganisms into a carrier fluid, for example the carrier fluid already mentioned above.

According to a further advantageous configuration, the reaction product can be limited to a space of less than 1000 μm in diameter. Since it is not necessary to culture the microorganisms before carrying out the method, it is possible to distinctly reduce the material requirements for carrying out the method. Moreover, this is advantageous because the substances required are frequently harmful to health or at least of concern to health.

According to a further advantageous configuration, the microorganisms tested can be retained on a filter material (e.g., a track-etched membrane) and the test can take place thereon and/or additionally it can be used for the concentration or deposition of the organisms from a relatively large sample volume. As a result, relatively large sample volumes can be introduced and reagents used can be saved, or used in a more precisely concentrated fashion, owing to the low sample volumes.

According to a further configuration, to quicken the performance of the method, the sample can be heated after addition of the at least one substance. In particular, the sample can be heated by 10° C., preferably by at least 10° C. or more. Fundamentally, it can be stated, however, that it is already possible to carry out the described method distinctly more rapidly, even without heating, compared to previously known detection methods. A classic culturing-based method, such as culturing on culture media/agar plates and use of contact plates, already requires several days, since this always requires culturing, which can be omitted with the claimed method.

According to a further advantageous configuration, as an alternative or in addition, the reaction product can be generated in a nutriment-free environment and/or in a culturing-free manner. It is therefore possible to ensure that unwanted propagation of microorganisms does not occur while carrying out the method.

According to a further advantageous configuration, the at least one specific substance used can be a profluorescent and/or a proluminescent and/or a prophosphorescent substance and/or a substance which brings about a color change. If these substances (substrates) are converted by proteins, such as specific enzymes as catalysts of the target organisms, they can be subsequently detected as reaction product. This detection can, for example, be performed by a cytometer, especially by a cytometer of a disk-shaped sample carrier (lab-on-a-chip). The sample carrier can be provided for microfluidics. It is, for example, possible to use profluorescent substrates having similar structures to ß-lactam antibiotics. If, for example, beta-lactamase-forming Gram-negative microorganisms, i.e., potentially antibiotic-resistant microorganisms, are present in a sample, they will convert the chosen substrate using their outer-membrane enzymes, such as beta-lactamases and/or carbapenemases. The reaction product is then measurable, and so the result provides information about whether the bacteria can degrade antibiotics. It is precisely in hospitals that such methods play an important role in being able to identify multiresistant pathogens early in order to contain their spread as quickly as possible. Fundamentally, the method according to the invention can, however, also be used for detecting other proteins or enzymes. Preferably, these can be proteins and/or enzymes which are on an outer side of a microorganism.

To be able to specify a critical analysis threshold, an optical detection limit can have been set such that the reaction product is only detected from a predetermined concentration. It is therefore possible to match the sensitivity of the method to the particular requirements as needed.

According to a further advantageous configuration, a step for determination of living microorganisms and/or dead microorganisms can be performed in the method. For example, this can be achieved by previously known substances (substrates), by which the living cells and/or dead cells can be stained for example. An optical evaluation for example is thus possible here, too. By contrast, previously known methods based on DNA/RNA probes have the disadvantage that no differentiation between the living cells and dead cells can be performed therewith. Such an embodiment can also be advantageous especially in combination with a configuration of measurement on the aforementioned filter material, since large volumes can be filtered in such a manner and, in this way, microorganisms can be retained on the filter material to be tested and they can be subsequently tested with respect to, for example, their vitality (living/dead/colony-forming/non-colony-forming). In relation to this, it may be useful to carry out an incubation of the organisms with or without a growth medium on the filter material or in a cavity of the fluidic system and to add a reagent which exclusively labels living and/or propagatable organisms. It is therefore possible to test a sample with respect to its sterility and/or concentration of living microorganisms.

According to a further development, to avoid possible escape of the reaction products and/or the microorganisms from a measurement loop, the sample which has been introduced and subjected to detection can be subsequently biologically deactivated, for example by autoclaving.

The invention also relates to a disk-shaped sample carrier comprising means for performance of a method as described and/or claimed herein. The sample carrier can, for example, be provided with microfluidics, for example with a channel system having microfluidic channels. This allows separate processing of individual microorganisms in a simple manner. In particular, the sample carrier comprises a receiving space for removal of the microorganisms from a sampling instrument and/or a nozzle for spraying of the microorganisms into a carrier fluid and/or a detection zone for quantitative optical detection of microorganisms, in the spatial proximity of which the reaction product is situated. The sample carrier has the advantage of being able to perform detection of a particular microorganism therewith on-site for example, such as in a hospital. It is therefore possible to carry out a rapid test at the protein level by the sample carrier. Special laboratory training or education for the user is not necessary. Moreover, due to the lack of propagation of the microorganisms in carrying out the method, the safety precautions to be taken in carrying out the method can be classified as distinctly lower than in the case of previously known methods.

One goal of the invention can be that of providing specific and rapid detection of properties of microorganisms such as, for example, the presence of carbapenemases, other beta-lactamases and/or a combination of multiple parameters by preferably handling-free detection within a lab-on-a-chip system. The system is intended for automatic processing and measurement of, for example, patient or hospital-environment samples. For example, it is intended here that ESBL-forming bacteria be specifically labeled and subsequently counted. It is desirable here to discriminate between living microorganisms and dead microorganisms. Differentiation between living and dead can, for example, be carried out on the basis of the presence or non-presence of enzyme activity. In addition, the method can be expanded by methods, such that the bacterial species is also determined. Thus, the result of an analysis can, for example, be: there is the presence of a carbapenemase and the organism “Acinetobacter baumanii” was identified. A hazard assessment is thus even distinctly better, simpler and quicker than was hitherto possible.

### BRIEF DESCRIPTION OF THE DRAWINGS

### Exemplary Embodiments

The invention will now be described in more detail on the basis of an exemplary embodiment, without being limited to said exemplary embodiment. Further exemplary embodiments arise from the combination of the features of individual or multiple claims with one another and/or with individual or multiple features of the exemplary embodiments

In the figures:

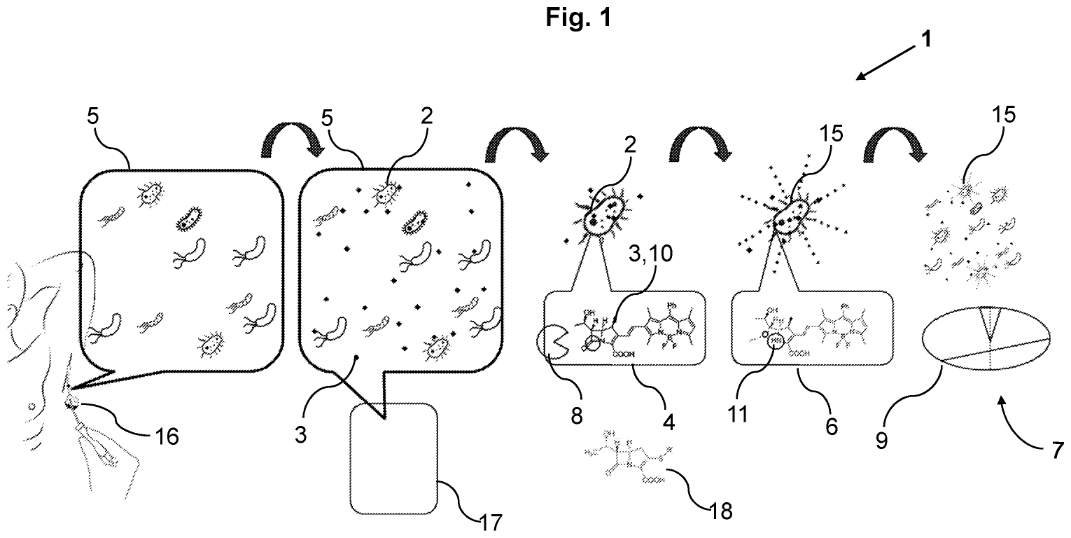

FIG. 1 shows a schematic depiction of a first variant of a method according to the invention, and

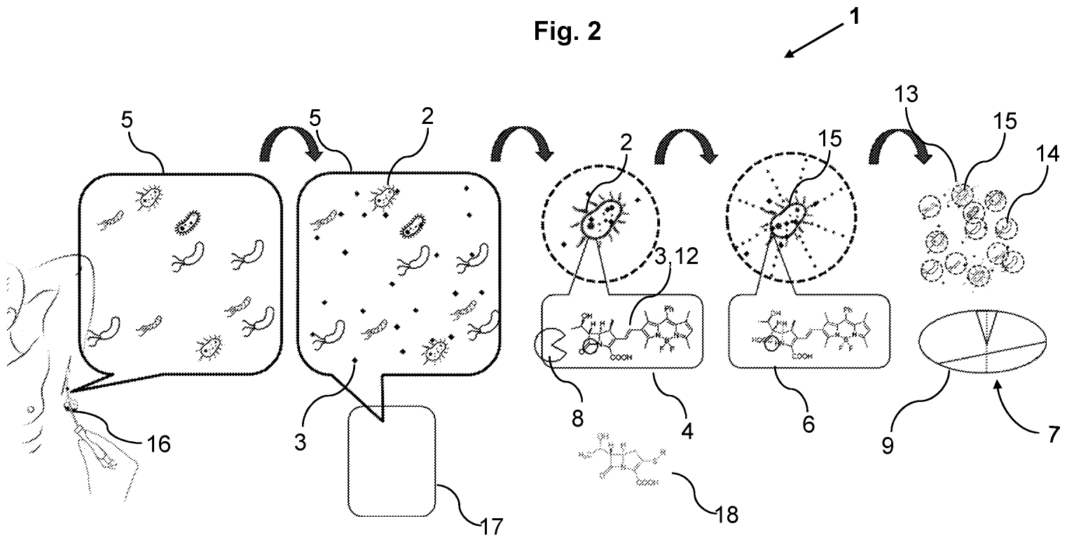

FIG. 2 shows a schematic depiction of a further variant of a method according to the invention.

### DETAILED DESCRIPTION

FIGS. 1 and 2 show different embodiments of a method for detecting microorganisms.

FIG. 1 shows a method 1 according to the invention for detecting microorganisms 2, wherein at least one specific substance 3 which triggers a reaction 4 of the microorganism 2 to be detected is added to a sample 5 in which the microorganisms 2 are to be detected. What are added as the specific substance 3 (in this case, a specific staining reagent) to a sample 5 to be tested are profluorescent and/or proluminescent and/or prophosphorescent substrates and/or substrates which bring about a color change. The specific substance 3 can be specifically bound to a particular antigen of the microorganism 2. Preferably, the specific substance 3 is bound to an outer side of the intact and/or living microorganism 2.

The reaction 4 involves generation of an optically detectable reaction product 6 which is subsequently detected in an optical detection method 7. The reaction product 6 is kept in spatial proximity to the reacting microorganism 2 until the optical detection 7.

If the aforementioned substrates (specific substance 3) are converted by at least one specific enzyme 8 as catalyst of the microorganisms 2 (target organisms), they are, for example, identifiable in a cytometer 9 (cell-counting and cell-analysis instrument), especially of a lab-on-a-chip system.

The substrate can, for example, be at least one profluorescent substrate having similar structures to ß-lactam antibiotics. If, for example, beta-lactamase-forming Gram-negative microorganisms, i.e., potentially antibiotic-resistant microorganisms, are present in a sample 5, they will convert the chosen substrate using their outer-membrane beta-lactamases.

The fluorescent reaction product 6 is fixed on the outer membrane of the microorganisms 2, for example by self-immobilizing substrates 10, which, after their conversion (and formation of a free-radical/reactive reactant 11), bind, for example, to the membrane structures/proteins of the microorganisms.

FIG. 2 shows a further variant of a method 1 according to the invention for detecting microorganisms 2, wherein at least one specific substance 3 which triggers a reaction 4 of the microorganism 2 to be detected is added to a sample 5 in which the microorganisms 2 are to be detected.

Furthermore, it is evident from FIG. 2 that what can be added as the specific substance 3 are substrates 12 which dissociate after their conversion. In this case, the microorganisms can, prior to analysis, be enveloped in droplets 13, 14 of small volume for local concentration of the signal. The droplets 13, 14 can, for example, have a volume of not more than 2000 μL, especially not more than 1500 μL, especially not more than 1000 μL, especially not more than 750 μL, especially not more than 500 μL, especially not more than 400 μL, especially not more than 300 μL, especially not more than 200 μL, especially not more than 100 μL.

Combination with other staining methods is also possible in order to detect different parameters, such as, for example, additional antibody labeling and/or staining with a dye for detection of living microorganisms and/or dead microorganisms.

Thereafter, the sample 5 is measured 7 by cytometry or other optical methods, such as, for example, imaging methods such as fluorescent microscopy, and the number of fluorescent and thus potentially antibiotic-resistant (especially living) microorganisms 15 is established.

The technology is likewise usable for the detection of other specific enzyme activities—especially if the products of the reaction are not present in the cell interior of the target organisms or dissociate.

The aforementioned example is based on an antibiotic-like substrate for detection of carbapenemase-forming organisms.

The invention is based on the proposal to use specific substrates in a lab-on-a-chip system in combination with local fixation of the signal for detection of, for example, antibiotic resistance.

In a preferred application, a sample 5 is first taken for carrying out the method. The sample 5 can, for example, be a sample which comes from a patient, such as a surface sample from a patient 16 and/or a blood sample and/or blood culture and/or urine sample.

The method is particularly suitable for carrying out a rapid test for the presence of an antibiotic-resistant pathogen, especially in a hospital, preferably on-site. In particular, the presence of multiresistant pathogens, such as multiresistant Gram-negative bacteria, can also be tested by the method.

The sample 5 is subsequently introduced into the sample carrier 17. The sample carrier 17 can, for example, be a sample carrier configured for microfluidics. Now, the sample 5 is automatically processed in the sample carrier 17, the sample 5 being combined with a specific substance 3, such as a profluorescent antibiotic-like dye 12, which is preferably bound to the outer side of the microorganisms.

The term specific substance can refer to the similarity to a particular antibiotic 18, for the resistance of which the microorganisms are to be tested, and is therefore specifically cut by an enzyme 8. In this connection, the microorganisms can be tested for multiple resistances through the use of multiple specific substances.

Thereafter, the detection reaction is carried out by optical signal capture 7 by cytometry. The detection reaction can also be carried out by other optical methods, such as, for example, imaging methods such as fluorescence microscopy. For example, the detection reaction can also be performed microfluidically. Antibiotic-resistant microorganisms express particular enzymes which, as catalysts, bring about the degradation and/or the inactivation of antibiotics. Said enzymes interact with the specific substance 3 (substrate; dye) and, for example, break them down. The result is a color reaction. The broken-down dye fluoresces near the microorganism or even in the microorganism, if it has been taken up. By optical evaluation 7, such as cytometry, in a cytometer 9, it is possible to determine the number of fluorescent and thus resistant microorganisms 15.

Because of a lack of culturing step, it is possible to carry out the method 1 even outside a laboratory, since the risk of contamination is very low. The samples 5 and/or the aforementioned droplets 13, 14 can therefore be free of growth medium.

The invention thus especially relates to a detection method 1 for antibiotic-resistant microorganisms 2, wherein at least one specific substance 3 which is broken down by an antibiotic resistance-causing enzyme 8 of the microorganism 2 is added to the sample 5, wherein the enzyme 8 present triggers a reaction 4, wherein the reaction 4 involves generation of an optically detectable reaction product 6 near the resistant microorganism 2 that is subsequently detected in an optical detection method 7.

### LIST OF REFERENCE SIGNS

1 Method according to the invention
2 Microorganism to be detected
3 Specific substance
4 A reaction of the microorganism to be detected 2
5 Sample containing microorganisms
6 Optically detectable reaction product
7 Optical detection
8 Specific enzyme of the microorganism 2
9 Cytometer
10 Self-immobilizing substrates
11 Free-radical/reactive reactant, formed after enzymatic conversion of the self-immobilizing substrates 10
12 Substrates which dissociate after their conversion
13 Droplets containing microorganism to be detected 2
14 Droplets containing microorganism not to be detected
15 Fluorescent microorganism to be detected
16 Surface sample of a patient
17 Sample carrier
18 Antibiotic

1. A method (1) for detecting at least one of microorganisms (2) or properties of the microorganisms, the method comprising:
adding at least one specific substance (3) which triggers a reaction (4) of the microorganism (2) to be detected to a sample (5) in which the microorganisms (2) are to be detected,
the reaction (4) resulting in involves generation of an optically detectable reaction product (6),
subsequently detected in optically detecting the optically detectable reaction product in an optical detection method (7), and
keeping the reaction product (6) is kept in spatial proximity to the reacting microorganism (2) until optical detection.

2. The method (1) as claimed in claim 1, wherein the reaction product (6) binds to an outer surface of the microorganism (2).

3. The method (1) as claimed in claim 1, wherein the individual microorganisms are surrounded by a phase boundary.

4. The method (1) as claimed in claim 1, further comprising adjusting a sensitive range of the detection method has been adjusted to a dimension of the microorganisms.

5. The method (1) as claimed in claim 1, further comprising generating or a phase boundary surrounding the individual microorganisms by spraying of the microorganisms into the or a carrier fluid.

6. The method (1) as claimed in claim 1, further comprising limiting the reaction product (6) to a space of less than 1000 μm in diameter.

7. The method (1) as claimed in claim 1, further comprising heating the sample (5) after addition of the at least one substance (3).

8. The method (1) as claimed in claim 1, wherein the at least one specific substance (3) used is at least one of a profluorescent substance, a proluminescent substance, a prophosphorescent substance, or a substance which brings about a color change.

9. The method (1) as claimed in claim 1, further comprising setting an optical detection limit such that the reaction product (6) is only detected from a predetermined concentration.

10. A disk-shaped sample carrier (17) configured to perform the method (1) as claimed in claim 1, the disk-shaped sample carrier comprising at least one of: a receiving space configured for removal of the microorganisms from a sampling instrument, a nozzle configured for spraying of the microorganisms into a carrier fluid, a detection zone configured for quantitative optical detection (7) of the microorganisms (2), in the spatial proximity of which the reaction product (6) is situated, or a filter material for retention or concentration of the microorganisms.

11. The method of claim 3, wherein the individual microorganisms are surrounded by the phase boundary in relation to an outer carrier fluid.

12. The method of claim 11, wherein the individual microorganisms are surrounded by the phase boundary in relation to a hydrophobic liquid.

13. The method of claim 3, wherein the reaction product (6) is enclosed by the phase boundaries.

14. The method of claim 7, wherein the heating is by at least 10° C.

15. The method of claim 7, wherein the reaction product (6) is generated in at least one of a nutriment-free environment or a culturing-free manner.

16. The method of claim 1, wherein the reaction product (6) is generated in at least one of a nutriment-free environment or a culturing-free manner.
